(19) United States
(12) Patent Application Publication
(10) Pub. No.: US 2009/0318531 A1
(43) Pub. Date: Dec. 24, 2009

(54) Small Interfering RNA Specific For HCV And Therapeutic Agent For Hepatitis C Comprising The Same

(75) Inventors: Meehyein Kim, Seoul (KR); Duckhyang Shin, Kyonggi-do (KR); Mahnhoon Park, Kyonggi-do (KR); Soo In Kim, Kyonggi-do (KR)

Correspondence Address: SUGHRUE MION, PLLC, 2100 PENNSYLVANIA AVENUE, N.W., SUITE 800, WASHINGTON, DC, 20037, US

(73) Assignee: Mogam Biotechnology Research Institute, Yongin-si (KR)

(21) Appl. No.: 11/915,975

(22) PCT Filed: May 30, 2006
(86) PCT No.: PCT/KR2006/002088

§ 371 Date: Nov. 29, 2007

(30) Foreign Application Priority Data
May 31, 2005 (KR) 1020050046392

(51) Int. Cl. A61K 48/00; C07H 21/02; C12N 15/63

(52) U.S. Cl. 514/44 A; 536/23.1; 435/320.1; 514/44 R

(57) ABSTRACT

The present invention relates to a therapeutic reagent for hepatitis C comprising HCV specific short interfering RNA (siRNA) as an effective ingredient. The siRNA of the invention is a double-stranded RNA specific for the nucleotide sequence of HCV which induces viral RNA degradation in mammalian cells and thereby inhibits HCV protein expression and replication. The method of the invention, which includes the step of administrating the synthetic siRNA or a DNA vector encoding the RNA, is thus effective for the treatment of HCV carrier by inhibiting HCV gene expression and replication.

### TECHNICAL FIELD

The present invention relates to a therapeutic agent for hepatitis C comprising HCV specific short interfering RNA (siRNA) as an effective ingredient.

### BACKGROUND ART

It is estimated that over 270 million people worldwide are chronically infected with Hepatitis C virus (HCV). From 40 to 60% of the patients with this viral pathogen may progress to liver cirrhosis or hepatocellular carcinoma (HCC). However, there is no vaccine available to protect its infection. One of the major anti-HCV therapies is treatment of alpha interferon (IFN-α) alone or in combination with ribavirin. But, current therapeutic regimens have limited efficacy and a poor response rate against certain HCV genotypes. Therefore, the development of new therapies to treat HCV infection is urgent.

RNA interference (RNAi) is evolutionally conserved process in which (endogenous and exogenous) gene expression is suppressed by introduction of double-stranded RNA (dsRNA) in all eukaryotes. RNAi is initiated by an RNase III-like endonuclease, called Dicer, which promotes consecutive cleavage of long dsRNAs into 21-23 nt short interfering RNAs (siRNAs) (Bernstein et al., Nature, 409: 363, 2001). The siRNAs are incorporated into an RNA-induced silencing complex (RISC), which unwinds the siRNAs in the presence of ATP (Hammond et al., Nature, 404: 293, 2000). The anti-sense RNAs incorporated into RISC recognizes the homologous RNAs and directs their degradation in the cellular cytoplasmic region.

The dsRNA over 30 nt in length induces a nonspecific interferon (IFN) response that activates protein kinase R (PKR) and RNase L (Balachandran et al., Immunity, 13: 129, 2000). The induction of PKR and RNase L activity finally leads to mRNA degradation and represses mRNA translation, nonspecifically, in mammalian cells. However, siRNAs are short enough to bypass the interferon pathway and directs gene silencing with sequence specificity (Elbashir et al., Nature, 411: 494, 2001). Generation of siRNA is expected to protect against genetic invasion caused by transposons, transgenes and viruses, which partially or completely harbor long dsRNA elements (Plasterk, Science, 296: 1263, 2002; Zamore, Science, 296: 1265, 2002; Hannon, Nature, 418: 244, 2002).

Many trials have been performed to select siRNAs to inhibit the replication of pathogenic RNA viruses, such as human immunodeficiency virus (HIV), hepatitis C virus (HCV), poliovirus, and so on (Novina et al., Nat Med, 8: 681, 2002; Wilson et al., Proc. Natl. Acad. Sci. USA, 100: 2783, 2003; Getlin et al., Nature, 418: 430, 2002). Especially, as sense strand RNA of HCV is related to not only synthesis of anti-sense strand RNA but also translation and viral assembly, it is a prime candidate for RNAi.

HCV is an enveloped virus and a member of the Flaviviridae family. Based on the nucleotide sequence composition, it is classified into at least six genotypes. The HCV genome is a positive-stranded ˜9.6 kb RNA consisting of a single open reading frame (ORF), which is flanked at the 5′ and 3′ ends by untranslated regions (UTRs). This ORF encodes a single long polyprotein that is approximately 3,000 aa in length, which is co- or posttranslationally processed into at least ten viral proteins: core, E1, E2, p7, NS2, NS3, NS4A, NS4B, NS5A, and NS5B (Bradley, Curr. Top Microbiol. Immunol., 242: 1, 2000; Reed and Rice, Curr. Top Microbiol. Immunol., 242: 55, 2000). For the viral replication cycle, negative-stranded RNA is de novo synthesized from viral genomic RNA by a replicase complex consisting of nonstructural (NS) proteins (Kim et al., J. Virol., 76: 6944, 2002). This intermediate negative-stranded RNA serves as a template for amplification of viral genomic RNAs that are translated into polyprotein of single ORF or encapsidated with viral structural proteins for mature viral particles.

Molecular and immunological studies of HCV replication have been hampered by the lack of a convenient animal model and available in vitro cell culture system. Although primary infection of the cultured cells with high-titer HCV-patient sera occurred, the condition of efficient viral amplification for detailed studies of HCV replication was not established. To circumvent this obstacle, selectable subgenomic RNAs, which replicate autonomously to high levels after transfection into the human hepatoma cell line Huh-7, has been developed (Lohmann et al., Science, 285: 110, 1999). The replicon of subgenomic HCV RNA lacking the structural region from C to p7 or NS2 was constructed by insertion of the selection marker gene, encoding neomycin phosphotransferase (neo), downstream of the HCV IRES, where as expression of the HCV NS proteins was directed by the IRES of the Encepholonyocarditis virus (EMCV). Significant progress in understanding HCV replication has been allowed by using this subgenomic replicon system or its genetically modified clones harboring adaptive mutations.

As there is an urgent need for development of an alternative treatment for HCV infection, HCV replicon system has been widely applied to develop HCV-specific siRNAs as anti-viral reagents (Randall et al., Proc. Natl. Acad. Sci. USA., 100: 235, 2003; Kapadia et al., Proc. Natl. Acad., Sci. USA., 100: 2014, 2003; Wilson et al., Proc. Natl. Acad. Sci. USA., 100: 2783, 2003; Yolota et al., EMBO Reports, 4: 602, 2003; Kronke et al., J. Virol, 78: 3436, 2004; Yuki et al., 2004, Microbiol. Immunol., 48, 591).

International Patent Publication Nos. WO/03/070750 and WO 2005/012525, and US Patent Publication No. US 2004/0209831 describe about siRNAs against HCV. But these siRNAs are not against the overall region of the HCV genome. To investigate the RNAi effect of the selected siRNAs, subgenomic HCV replicon RNAs not containing structural region from C to p7 has been applied as a useful screening tool. However, it is required to select the effective SiRNAs systematically against the overall region of the HCV RNA genome.

In the present invention, the inventors introduced the cell-based replicon system with full-length HCV RNA and assessed their efficacy to inhibit viral replication and expression. As a result, the present inventors completed this invention by confirming that an siRNA having specific sequence could efficiently inhibit HCV protein expression and RNA synthesis.

### DISCLOSURE

### Technical Problem

It is an object of the present invention to provide a HCV specific siRNA, an expression vector expressing the siRNA and a pharmaceutical composition for the treatment and prevention of hepatitis C comprising the siRNA or the siRNA expression vector as an effective ingredient.

### Technical Solution

To achieve the above object, the present invention provides a nucleic acid molecule comprising one of the nucleotide sequences of SEQ. ID. NOs: 1-36, or a complement thereof, or a portion thereof.

The present invention also provides an expression vector expressing the siRNA.

The present invention further provides a pharmaceutical composition for the treatment and prevention of hepatitis C comprising the siRNA or the siRNA expression vector as an effective ingredient.

The present invention also provides a treatment method for HCV infection related disease comprising the step of injecting the siRNA or the siRNA expression vector into a subject.

Hereinafter, the present invention is described in detail.

The present invention provides an isolated nucleic acid molecule comprising one of the nucleotide sequences of SEQ. ID. NOs: 1-36, or a complement thereof, or a portion thereof.

In a preferred embodiment, siRNA is obtained by hybridization of the two complementary synthetic RNAs or delivery of a vector encoding the RNA in the cell. For efficient inhibition of the viral replication, target sequences of the segments on the HCV full-length RNA were selected as SEQ. ID. NOs: 1-36.

(SEQ. ID. NO: 1)

siHCV-55: 5′-CUACUGUCUUCACGCAGAA-3′;

(SEQ. ID. NO: 2)

siHCV-74: 5′-AGCGUCUAGCCAUGGCGUU-3′;

(SEQ. ID. NO: 3)

siHCV-207: 5′-CCCGCUCAAUGCCUGGAGA-3′;

(SEQ. ID. NO: 4)

siHCV-523: 5′-GGCGACAACCUAUCCCCAA-3′;

(SEQ. ID. NO: 5)

siHCV-704: 5′-GGUCAUCGAUACCCUCACG-3′;

(SEQ. ID. NO: 6)

siHCV-845: 5′-UCUGCCCGGUUGCUCCUUU-3′;

(SEQ. ID. NO: 7)

siHCV-929: 5′-CGUAUCCGGAGUGUACCAU-3′;

(SEQ. ID. NO: 8)

siHCV-968: 5′-CGCAAGCAUUGUGUAUGAG-3′;

(SEQ. ID. NO: 9)

siHCV-1264: 5′-UAUAUCCCGGCCACGUGAC-3′;

(SEQ. ID. NO: 10)

siHCV-1631: 5′-UGACUCCCUCAACACUGGG-3′;

(SEQ. ID. NO: 11)

siHCV-1980: 5′-GGCAACUGGUUUGGCUGUA-3′;

(SEQ. ID. NO: 12)

siHCV-2486: 5′-AUGGGAGUAUGUCCUGUUG-3′;

(SEQ. ID. NO: 13)

siHCV-3013: 5′-UCUUGCUCGCCAUACUCGG-3′;

(SEQ. ID. NO: 14)

siHCV-3061: 5′-CCAAAGUGCCGUACUUCGU-3′;

(SEQ. ID. NO: 15)

siHCV-3122: 5′-GGUUGCUGGGGGUCAUUAU-3′;

(SEQ. ID. NO: 16)

siHCV-7284: 5′-GGCACAUGGUAUCGACCCU-3′;

(SEQ. ID. NO: 17)

siHCV-7796: 5′-UGACGGGCUUUACCGGCGA-3′;

(SEQ. ID. NO: 18)

siHCV-8373: 5′-CGAGGUUACUACCACACAC-3′;

(SEQ. ID. NO: 19)

siHCV-8504: 5′-CAGGCAGCGUGGUCAUUGU-3′;

(SEQ. ID. NO: 20)

siHCV-8546: 5′-AGCCGGCCAUCAUUCCCGA-3′;

(SEQ. ID. NO: 21)

siHCV-8572: 5′-GUCCUUUACCGGGAGUUCG-3′;

(SEQ. ID. NO: 22)

siHCV-8672: 5′-UCGGGUUGCUGCAAACAGC-3′;

(SEQ. ID. NO: 23)

siHCV-8749: 5′-GCCUUCUGGGCGAAGCAUA-3′;

(SEQ. ID. NO: 24)

siHCV-9117: 5′-CCUACUCCCUGCUAUCCUC-3′;

(SEQ. ID. NO: 25)

siHCV-9650: 5′-CAUUCCCCAUUAACGCGUA-3′;

(SEQ. ID. NO: 26)

siHCV-10464: 5′-GAGGACGGUUGUCCUGUCA-3′;

(SEQ. ID. NO: 27)

siHCV-10486: 5′-UCUACCGUGUCUUCUGCCU-3′;

(SEQ. ID. NO: 28)

siHCV-10881: 5′-GAAGGUCACCUUUGACAGA-3′;

(SEQ. ID. NO: 29)

siHCV-11048: 5′-AGGACGUCCGGAACCUAUC-3′;

(SEQ. ID. NO: 30)

siHCV-11196: 5′-GCCAGCUCGCCUUAUCGUA-3′;

(SEQ. ID. NO: 31)

siHCV-11476: 5′-GCCAGACAGGCCAUAAGGU-3′;

(SEQ. ID. NO: 32)

siHCV-11796: 5′-ACCAGAAUACGACUUGGAG-3′;

(SEQ. ID. NO: 33)

siHCV-11996: 5′-GGAUGAUCCUGAUGACUCA-3′;

(SEQ. ID. NO: 33)

siHCV-12509: 5′-CGGGGAGCUAAACACUCCA-3′;

(SEQ. ID. NO: 35)

siHCV-12520: 5′-ACACUCCAGGCCAAUAGGC-3′;
and

(SEQ. ID. NO: 36)

siHCV-12680: 5′-AGGUCCGUGAGCCGCUUGA-3′.

To increase the stability of synthetic siRNA or the specific interaction between viral target RNA and siRNA Fragment, the 3′-ends of both strands of siRNA were extended with dTdT via chemical synthesis. In a preferred embodiment, synthetic siRNA can be modified internally with chemical derivatives or tagging-molecules for acquiring its physiological stability and chasing its distribution in cells.

The invention also demonstrates the RNAi activity induced by synthetic siRNAs in which 3′-end of each strand RNA in extended with dTdT for their stability. The synthetic RNAs efficiently inhibit gene expression by ˜80% in the replicon cell line. It will be a new therapeutic approach for treating a hepatitis viral carrier, infected by HCV, by administration to a subject in need thereof the synthetic siRNA or the vector.

The present invention also provides an expression vector for the siRNA expression.

In a preferred embodiment, shRNA (short hairpin RNA) can be transcribed from a single promoter independently and processed into double-stranded siRNA by cellular Dicer. Alternatively, each strand of double-stranded siRNA is expressed from the two separated promoters, in opposite or in parallel, and hybridizes following induction of degradation of target RNAs. A vector expressing siRNA contains not only promoter(s) for initiation of transcription but also enhancer, transcription termination signal, or other expression regulatory sequences. The vector can be delivered into the cellular nucleus as a form of naked plasmid DNA, complex with transfection reagent or target-delivery material, or recombinant viral vector. The construction of the vector is determined by specific situations, such as the cell state or type to be transfected, the time and level of siRNA expression, and so on.

The present invention provides a vector pRNAiDu for direct expression of siRNA, which is transcribed from convergent opposing promoters (see FIG. 1). The vector contains two convergent RNA polymerase III promoters (H1 and U6) and SV40 promoter inducing the transcription of mRNA encoding EGFP-Fluc. Specially, in the pRNAiDu vector, the fusion gene of enhanced green fluorescent protein (EGFP) and firefly luciferase (Fluc), EGFP-Fluc, is contained under the SV40 promoter. Experimentally, this is useful to visualize and quantitatively monitor the transfection efficiency, and to standardize the RNAi activity via detection of fluorescence or luminescence (Kaykas and Moon, BMC Cell Biology, 5: 16, 2004; Zheng et al., Proc Natl Acad Sci USA, 101: 135, 2004).

The present inventors have deposited the E. coli transformed with the expression vector at Korean Collection for Type Cultures (KCTC) of Korea Research Institute of Bioscience and Biotechnology (KRIBB) on May 16, 2005 (Accession No: KCTC 10800BP).

To increase the stability of synthetic siRNA or the specific interaction between target RNA and siRNA fragment, the 3′-ends of both strands of siRNA were extended with dTdT via chemical synthesis. In the siRNA expression vector, two nucleotides of UU at the 3′-ends are generated from polymerase III termination sequences at the final stage of RNA transcription. RNA interference (RNAi) effect is dependent on the detection time and transfected DNA dose and causes lower than 50% and 60% of inhibition of protein expression and viral RNA transcription level, respectively. As the amount of enhanced green fluorescent protein (EGFP) expressed from the siRNA vector presents 50% of the transfection efficiency by FASC analysis, it can be considered that the selected siRNAs induce dramatic and specific anti-viral effect. Especially, the siRNA expression cassette, separated from the vector by PCR amplification with 5′-phosphorylated primers, is the essential element inducing the RNAi effect.

The present invention further provides a pharmaceutical composition for the treatment and prevention of hepatitis C comprising the siRNA or the expression vector as an effective ingredient.

This invention is based on the discovery short interfering RNA (siRNA) molecules by targeting Hepatitis C virus (HCV) RNA based on the full-length replicon RNA, FK/R2AN, that is stably maintained in Huh-7 cell line. The viral specific siRNAs induce degradation of this exogeneous RNA, and finally inhibits expression of viral proteins and the viral replication in mammalian cells. This suggests that the siRNA or the expression vector can be included in a therapeutic composition for hepatitis C as an effective ingredient.

An siRNA of the present invention can be synthesized chemically or enzymatically (Caruthers et al., Methods in Enzymology, 211: 3, 1992; Wicott et al., Nucleic Acids Res, 23: 2677, 1995; Brennan et al., Biotechnol Bioeng, 61: 33, 1998).

An siRNA or vector of this invention can be delivered to a target cells using by transfection-materials, such as liposomes, hydrogels, bioadhesive microspheres and the like (Akhtar et al., Trends Cell Bio, 2: 139, 1992).

A pharmaceutical composition contains an siRNA or vector of this invention with an organ targeting material and a pharmaceutically acceptable carrier for treating an infection with HCV. The dose of pharmaceutical composition can be determined, therapeutically, by a specific situation, such as the route of administration, the nature of the formulation, the phase of liver failure, the subject's size, weight, or distribution range, and the age and sex of patient.

The therapeutic composition of the invention includes the effective ingredient by 0.0001-50 weight % for the total weight of the composition.

The composition of the present invention can additionally include one or more effective ingredients having the same or similar functions to the active ingredient.

The composition of the present invention can also include, in addition to the above-mentioned effective ingredients, one or more pharmaceutically acceptable carriers for the administration. Pharmaceutically acceptable carrier can be selected or be prepared by mixing more than one ingredients selected from a group consisting of saline, sterilized water, Ringer's solution, buffered saline, dextrose solution, maltodextrose solution, glycerol, ethanol and liposome. Other general additives such as anti-oxidative agent, buffer solution, bacteriostatic agent, etc, can be added. In order to prepare injectable solutions, pills, capsules, granules or tablets, diluents, dispersing agents, surfactants, binders and lubricants can be additionally added. The composition of the present invention can further be prepared in suitable forms for each disease or according to ingredients by following a method represented in Remington's Pharmaceutical Science (the newest edition), Mack Publishing Company, Easton Pa.

The composition of the present invention can be administered orally or parenterally (for example, intravenous, hypodermic, local or peritoneal injection) The effective dosage of the composition can be determined according to weight, age, gender, health condition, diet, administration frequency, administration method, excretion and severity of a disease. The dosage of the composition of the invention is 0.1˜100 mg/kg per day, and preferably 0.5˜10 mg/kg per day. Administration frequency is once a day or preferably a few times a day.

The siRNA or the siRNA expression vector of the present invention was intravenously injected into mice to investigate toxicity. As a result, it was evaluated to be safe substance since its estimated LD₅₀ value is much greater than 1,000 mg/kg in mice.

The present invention also provides a treatment method for HCV infection related disease comprising the step of injecting the siRNA or the siRNA expression vector into a subject.

This invention demonstrates a therapeutic application of synthetic siRNA or vector encoding double-stranded siRNA and the combination therapy containing siRNA to inhibit HCV replication in viral carriers.

### DESCRIPTION OF DRAWINGS

The application of the preferred embodiments of the present invention is best understood with reference to the accompanying drawings, wherein:

FIG. 1 is a schematic diagram of the pRNAiDu plasmid.

FIG. 2 is a schematic diagram of the siRNA expression cassette constructed for the expression of HCV-specific siRNA.

FIG. 3 is a schematic diagram of the full-length HCV replicon, FK/R2AN.

FIG. 4 and FIG. 5 are diagrams showing the effect of RNAi on HCV replication in FK/R2AN cells.

FIG. 4 is a graph illustrating that the HCV full-length replicon, FK/R2AN, cells were transfected with siRNA vectors expressing the positive control, HCV-specific, or Rluc siRNA and tested by Dual Luciferase Assay.

FIG. 5 is a graph illustrating that the HCV full-length replicon cells were transfected with siRNA expression cassettes generated by PCR amplification and tested by Renilla Luciferase Assay.

FIG. 6 is a graph showing the results of real-time RT-PCR analysis of HCV replicon RNAs in cells transfected with siRNA expression vectors.

FIG. 7 and FIG. 8 represent the RNAi effects on HCV replicon, FK/R2AN, by synthetic siRNAs.

FIG. 7 is a photograph showing the results of Western blot analysis using monoclonal antibodies specific for HCV core and β-actin.

FIG. 8 is a graph showing the results of Rluc assay illustrating the expression level of the reporter protein in FK/R2AN cells treated with synthetic siRNA to confirm the RNAi activities of selected siRNA candidates.

FIG. 9 is a graph showing the viral RNA levels of the FK/R2AN cells delivered with chemically synthesized siHCV-7284.

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### siRNA and siRNA Expression Vector

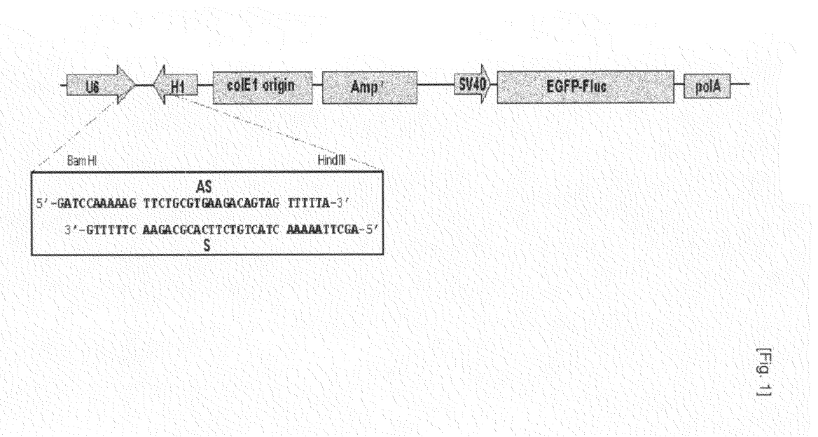

In mammalian cells, previously siRNA vectors have been designed to transcribe short hairpin RNAs (shRNAs) from an RNA polymerase III promoter (such as U6, H1, and tRNA promoters) or a polymerase II promoter with a poly(A) signal sequence (Brummelkamp et al., Cancer Cell, 2: 243, 2002; Tushcl, Nat. Biotechnol., 20: 446, 2002; Xia et al., Nat. Biotechnol., 20: 1006, 2002). However, shRNA vectors show multiple drawbacks. Their non-natural secondary structure induces that it is hard to synthesize them in bacteria and to sequence, and DNA oligomers to generate them can be costly in the case of high through-put screening. Moreover, it is less facile to generate an siRNA expression cassette containing a promoter to a termination signal without additional sequences for constructing diverse siRNA library. To circumvent these limitations of shRNA expression vectors, the present inventors constructed a vector for direct expression of siRNA, which is transcribed from convergent opposing promoters, and named it pRNAiDu (FIG. 1).

The present inventors have deposited the E. coli transformed with the expression vector at Korean Collection for Type Cultures (KCTC) of Korea Research Institute of Bioscience and Biotechnology (KRIBB) on May 16, 2005 (Accession No: KCTC 10800BP).

The vector contains two convergent RNA polymerase III promoters (H1 and U6) and SV40 promoter inducing the transcription of mRNA encoding EGFP-Fluc. Specially, in the pRNAiDu vector, the fusion gene of enhanced green fluorescent protein (EGFP) and firefly luciferase (Fluc), EGFP-Fluc, is contained under the SV40 promoter. Experimentally, this is useful to visualize and quantitatively monitor the transfection efficiency, and to standardize the RNAi activity via detection of fluorescence or luminescence (Kaykas and Moon, BMC Cell Biology, 5: 16, 2004; Zheng et al., Proc Natl Acad Sci USA, 101: 135, 2004).

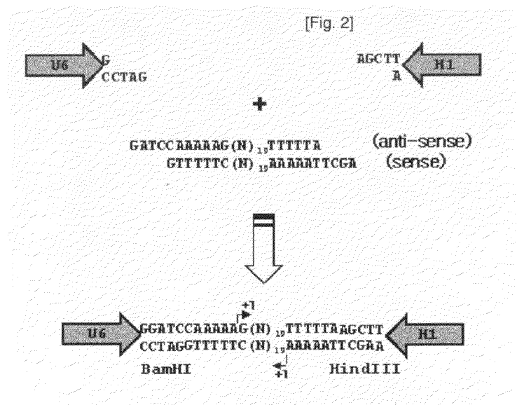

Both the human U6 and H1 promoters were modified to contain a RNA polymerase III termination sequences of five thymidine nucleotides at the −5 to −1 position and a BamH I site and a Hind III site at each −12 to −6 position, respectively. As the U6 promoter prefers a purine nucleotide for efficient transcription initiation, guanidine is inserted at the +1 position downstream of the U6 promoter in the case of that the first nucleotide sequence of siRNA is a pyrimidine. To minimize an artificial effect induced this additional nucleotide and guarantee a consecutive hybridization between anti-sense siRNA and target RNA in the RNAi process, it was devised that the U6 promoter takes a charge of transcription for the anti-sense RNA, which directs RISC to cleave the homologous mRNA. To create the siRNA expression plasmids, pairs of 36-base oligonucleotides were annealed and ligated into pRNAiDu digested with BamH I and Hind III (FIG. 2). In the FIG. 2, initiation sites of siRNA transcription are indicated with arrows.

Isolation of siRNA expression cassette from U6 and H1 promoter was performed by PCR amplification. Both U6 forward primer (5′-CGGAATTCCCCAGTGGAAAGAC-3′: SEQ. ID. NO: 37) and H1 forward primer (5′-CGGAATTCATATTTGCATGTCGC-3′: SEQ. ID. NO: 38) were prepared by modification at 5′-end of each oligonucleotide with phosphate. The individual siRNA expression cassettes were amplified from the corresponding pRNAiDu plasmids containing HCV-specific, negative control, or positive control siRNA sequence. All PCR reactions were carried out as follows: 45 sec at 94° C. 1 min at 50° C., and 1 min at 72° C. for 30 cycles. The PCR products were purified using the Qiaquick PCR purification kit (Qiagen, USA) and their concentration was quantitatively measured by using UV-spectrophotometer.

### Example 2

### Inhibition of Protein Expression of FK/R2an Replicon by HCV Specific siRNAs

The Huh-7 cell line and its replicon cell line that stably supports HCV full-length replicon RNA of genotype 1b (Huh-7FK/R2AN) were obtained from Dr. S. K. Jang (PanBioNet, Korea). The FK/R2AN replicon is a modification from the HCV full-length replicon RNA, FK/RN (Korean Patent Publication No. 2004-32341), with the insertion of the signal sequence of foot-and-mouth disease virus (FMDV) 2A protease, which locates between Rluc and neo coding regions. Cell lines carrying HCV replicons were grown in DMEM containing 10% FBS (Gibco, USA) and 600 mg/ml G418 (Calbiochem, USA) and used in all experiments.

Transfection of DNAs of the siRNA expression plasmids or PCR products, or synthetic siRNA oligonucleotides was performed in 12-well plate using Lipofectamine 2000 reagent (Invitrogen, USA) in accordance with the manufacturer's instructions. Briefly, 1.5×10⁵ Huh-7 FK/R2AN cells were plated, and the following day, 0.6 mg of siRNA expression DNA or 80 nM of siRNA duplexes were delivered. Renilla luciferase (Rluc) activity expressed from replicon RNA as a reporter protein was adjusted using firefly luciferase (Fluc) activity expressed from the transfected DNA vector, or corrected by total protein amount in the case of transfection with PCR products or synthetic siRNAs to normalize the transfection efficiency.

Total cellular proteins in replicon cells were harvested at day 3 posttransfection and luciferase activities were quantified using the Dual-Glo Luciferase Assay System (Promega, USA) or the Renilla Luciferase Assay System (Promega, USA) to monitor indirectly the RNAi effect by HCV-specific siRNAs.

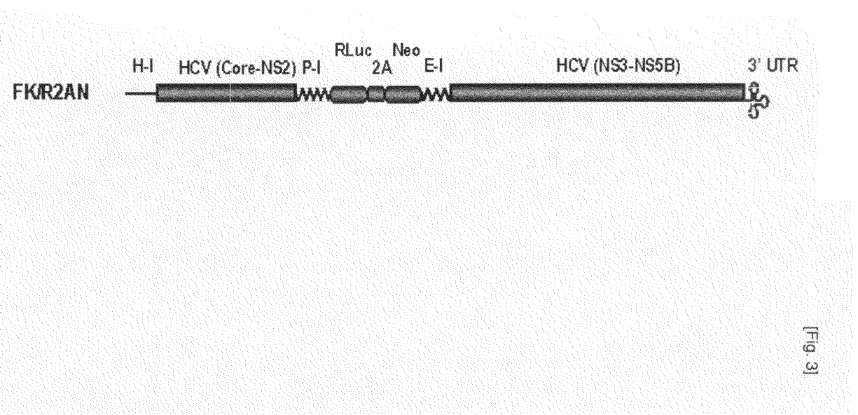

To assess the siRNA-mediated RNAi effects on HCV replication in cellular cytoplasm, the present inventors utilized HCV full-length HCV replicon system. In this HCV replicon RNA, FK/R2AN, mRNA sequences of Rluc and neo were introduced between HCV NS2 and NS3 coding regions and expressed into proteins by direction of the polio virus IRES. This fusion protein is fated to be processed into separate proteins by cleavage at the signal sequence of foot-and-mouth disease virus (FMDV) 2A protease, which locates between Rluc and neo coding regions. This finally enables the selection of cells which stably maintain the replicon RNA and its expression, and also enables the precise quantification of replication levels by monitoring luciferase activity of the cell lysates (FIG. 3). H-I, HCV internal ribosomal entry site (IRES); Core-NS2, HCV coding region from core to NS2; P-I, poliovirus IRES; Rluc, renilla luciferase gene; 2A, foot-and-mouth disease virus (FMDV) 2A protease cleavage site; Neo, neomycin phosphotransferase gene; E-I, encephalomyocarditis virus (EMCV) IRES; NS3-NS5B, HCV coding region from NS3 to NS5B, 3′ UTR, HCV 3′ untranslated region.

To selection HCV-specific siRNAs with efficient anti-viral effect, we designed 36 HCV siRNA candidates, one negative control RNA which was isolated from commercially available siRNA expression vectors (Ambion, USA), or a Rluc specific siRNA which was screened experimentally as an active sequence in our laboratory from three candidates.

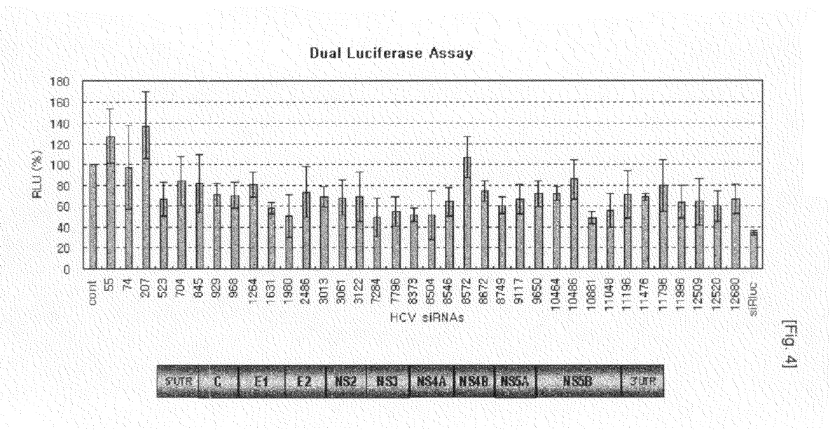

To monitor the silencing effects of these 38 siRNAs, one for negative control and others against HCV replicon RNA, the present inventors prepared siRNA expression plasmids in which siRNAs are transcribed from convergently opposing human U6 and H1 promoters, and also produced the siRNA expression cassettes by PCR amplification. Both of the individual DNAs were transfected into FK/R2AN replicon cells, independently, and the replication levels affected by two different siRNA expression systems against replicon RNA were compared by measuring Rluc activity (FIG. 4 and FIG. 5).

FIG. 4 and FIG. 5 are graphs showing the effect of RNAi on HCV replication in FK/R2AN cells. The HCV full-length replicon, FK/R2AN, cells were transfected with siRNA vectors expressing the positive control, HCV-specific, or Rluc siRNA. The internal renilla luciferase activities were measured at day 3 after transfection and normalized by firefly luciferase activity directed from transfected DNA vectors, by dual luciferase assay. The HCV full-length replicon cells were transfected with siRNA expression cassettes generated by PCR amplification. Values are shown as percentages of the siRNA negative control, as the mean±s.d. The genetic positions of siRNA targets on the replicon RNA were presented schematically. Representative data from at least three independent experiments are shown.

As shown in FIG. 4 and FIG. 5, suppression profiles for this reporter gene expression resulted from transfection of plasmids into FK/R2AN cells closely matches those from transfection of equivalent amount of PCR products which contain genetically essential element for directing RNAi effect. Of the 38 triggers tested, siRNAs of siHCV-523, 1631, 7284, 8373, 8504, 8794, 11048, 11996, and 12509 elicited the most potent effect, in common. Especially, in the vector-based system, the most potent HCV siRNAs reduced the protein expression by ˜35-50% (FIG. 4). As considering that the transfection efficiency of these plasmid DNAs into FK/R2AN cells using lipofectamine 2000 reagents (Invitrogen, USA) reaches approximately 50% by FACS analysis, these suppression levels demonstrate that the viral RNAs distributed in the cellular cytoplasm can be totally eradicated by siRNA-induced silencing pathway.

The vector-based siRNAs inhibited the expression of the HCV replicon more potently than the PCR-product-based siRNAs did (FIG. 5). It might be caused by the high transfection efficiency and the increased cellular viability of circular DNAs compared with PCR products of linear DNAs. The levels of suppression of the replicon by HCV-specific siRNA expression vectors as compared with PCR products, respectively, were: 33% compared with 35% for siHCV-523; 42% compared with 18% for siHCV-1631; 51% compared with 44% for siHCV-7284; 48% compared with 26% for siHCV-8373; 49% compared with 34% for siHCV-8504; 39% compared with 36% for siHCV-8749; 44% compared with 17% for siHCV-11048; 36% compared with 23% for siHCV-11996; 36% compared with 18% for siHCV-12509; and 66% compared with 67% for siRluc. The clones of siRNA expression vectors were applied for further investigation of gene silencing effects by alternative approaches.

In this invention, the present inventors prepared three different plasmids harboring HCV 5′UTR-specific siRNAs, because this region has been considered to be the most ideal region for development of siRNA therapeutics. However, the inventors observed no significant suppression of an HCV genomic replicon by these siRNAs. It can be predicted that its secondary structure is too stable for siRNAs incorporated with RISC to approach the target region via unwinding and replacement of Watson-Crick base pairs, or cellular or viral proteins flock together around this region to regulate the viral gene expression and encapsidation and result in the siRNA-targeting unfavorable.

### Example 3

### Reduction of Viral Transcripts by Vector-Mediated HCV siRNAs

Total RNA was extracted from FK/R2AN cells transfected with negative control or HCV replicon-specific siRNA expression vectors, at day 2 posttransfection, using Trizol LS reagent (Invitrogen, USA) according to the manufacturer's instruction. The isolated total RNA was digested with RNase-free DNase (Promega, USA). Finally, absolute amount of RNA was quantified by measuring UV-absorbance at 260 nm/280 nm using UV-spectrophotometer.

In vitro antiviral activity was assessed by means of a quantitative real-time RT-PCR (Sequence Detection System 5700; Applied Biosystems, USA). The real-time RT-PCR was performed with 500 ng of total RNAs isolated from the transfected cells in a reaction volume of 50 μl using the TaqMan One-Step RT-PCR Master Mix Reagents (Applied Biosystems, USA). Standard RNA was prepared by in vitro transcription of 5′ UTR RNA by T7 RNA polymerase. The primer and probe sequences, specific for HCV 5′UTR region, include 5′-TCTGCGGAACCGGTGAGTA-3′ (forward primer; SEQ. ID. NO: 39), 5′-ATTGAGCGGGTTGATCCAAGAAA-3′ ((reverse primer; SEQ. ID. NO: 40) and 5′-(fluorescein) CCGGAATTGCCAGGACGACCG (TAMRA)-3′ (probe; SEQ. ID. NO: 41). The total RNA amount was adjusted, definitely, by carrying out real-time RT-PCR targeting human-actin gene as an internal control, in parallel. The primer and probe sequences for -actin gene include 5′-GCGCGGCTACAGCTTCA-3′ (forward primer; SEQ. ID. NO: 42), 5′-TCTCGTTAATGTCACGAT-3′ (reverse primer; SEQ. ID. NO: 43) and 5-(fluorescein) CACCACGGCCGAGCGGGA (TAMRA)-3′ (probe; SEQ. ID. NO: 44). All experiments were performed in triplicate.

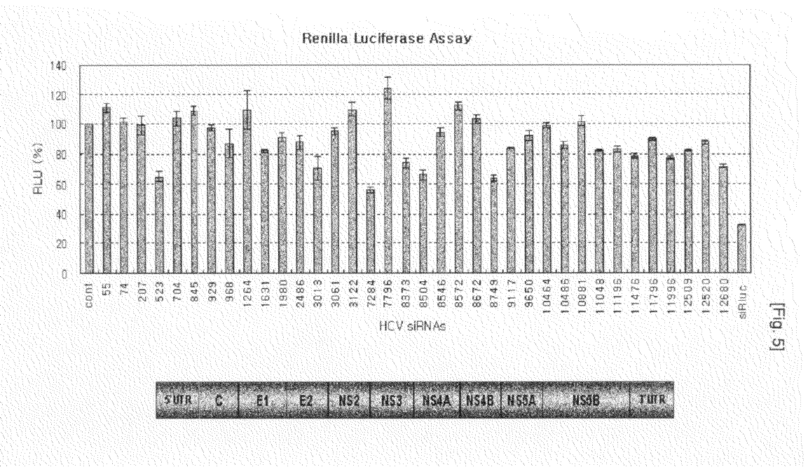

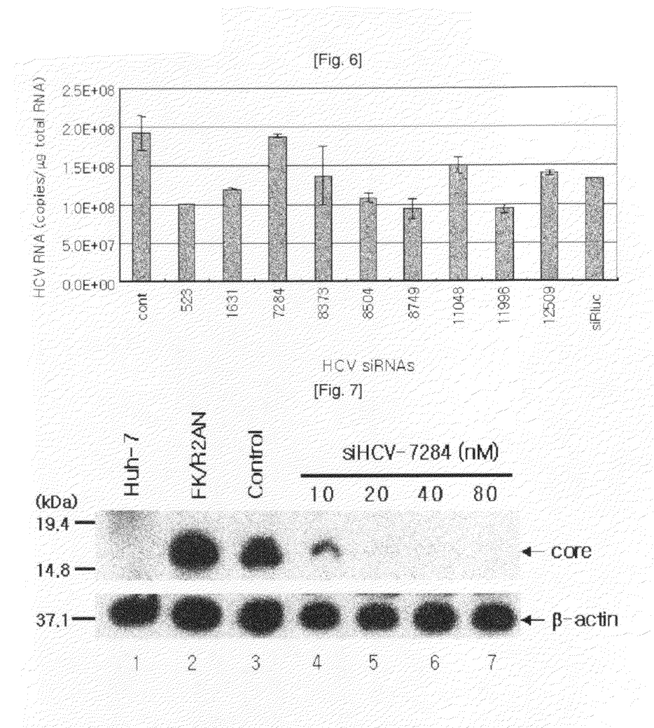

To accurately quantify the HCV RNA levels in FK/R2AN cells transfected with selected siRNA expression vectors, the present inventors performed real-time RT-PCR by using primers specific for HCV 5′ UTR and -actin genes (FIG. 6).

FIG. 6 is a graph showing the results of real-time RT-PCR analysis of HCV replicon RNAs in cells transfected with siRNA expression vectors. The FK/R2AN cells were delivered with 600 ng of pRNAiDu plasmids which express negative control, HCV-specific, or Rluc siRNA in 12 well plates. For quantitative analysis, HCV RNA values were normalized to -actin RNA values. The numbers of HCV RNA copies per microgram of total RNA were estimated. Representative data (mean±s.d.) are presented from at least three independent experiments.

As shown in FIG. 6, quantitative analysis revealed that HCV transcript levels were decreased by 62% in cells transfected with siHCV-8749 or 11996, and 47% with siHCV-523, respectively, on day 2 posttransfection. Similar inhibition profile was obtained by measuring RNA levels in transfection experiments with PCR product-based siRNA cassettes. These results demonstrate that the selected HCV siRNAs showing the greatest inhibition effect in protein levels also potently decreased replicon RNA levels by inducing viral RNA degradation. Unexpectedly, siHCV-7284 clone showed no significant reduction in viral RNA levels. The present inventors speculate that the amount of individual siRNAs produced from the siRNA expression vectors might not be identical at various times and should be affected by their transcription efficiency and the physiological stability in cells.

### Example 4

### Detection of RNAi Effects by Synthetic siRNAs

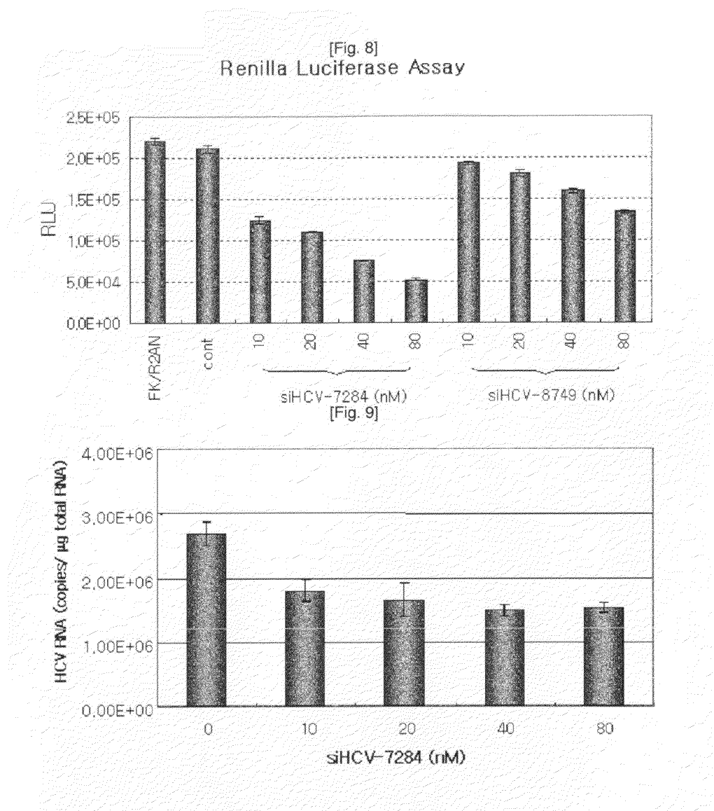

The present inventors prepared synthetic sense and anti-sense siRNAs and annealed them to duplex RNAs corresponding to control siRNA, siHCV-7284 and siHCV-8749, representatively. Their nucleotide sequences are as follows: sense strand of control siRNA, 5′-ACUACCGUUGUUAUAGGUGTT-3′ (SEQ. ID. NO: 45); anti-sense strand of control siRNA, 5′-CACCUAUAACAACGGUAGUTT-3′ (SEQ. ID. NO: 46); sense strand of siHCV-7284, 5′-GGCACAUGGUAUCGACCCUTT-3′ (SEQ. ID. NO: 47); anti-sense strand of siHCV-7284, 5′-AGGGUCGAUACCAUGUGCCTT-3′ (SEQ. ID. NO: 48); sense strand of siHCV-8749, 5′-GCCUUCUGGGCGAAGCAUATT-3′ (SEQ. ID. NO: 49; anti-sense strand of siHCV-8749, 5′-UAUUCGCCCAGAAGGCTT-3′ (SEQ. ID. NO 50). FK/R2AN cells were treated with equivalent concentration of 80 nM siRNAs and harvested for quantification of RNA transcript levels by real-time RT-PCR at day 2 and for measuring replicon protein levels by luciferase activity assay or Western blot assay at day 3 after transfection. The concentrations of all transfections were brought up to a final concentration of 80 nM with control siRNA. All experiments were performed at least in triplicate (FIG. 7 and FIG. 8).

FIG. 7 and FIG. 8 show the RNAi effects on HCV replicon, FK/R2AN, by synthetic siRNAs. FIG. 8 is a set of photographs illustrating the results of Western blot analysis using monoclonal antibodies specific for HCV core and -actin. Western blot analysis was performed with 30 mg total proteins of cell lysates from synthetic siRNA-treated FK/R2AN. Proteins were separated using 15% SDS-PAGE and transferred to Immobilon-P membrane (Millipore, USA). The blots were probed with monoclonal antibodies specific for HCV core (Affinity BioReagent™, USA) and -actin (Sigma, USA), followed by incubated with a horseradish peroxidase-conjugated goat anti-mouse antibody (KPL, USA). The blots were developed with Super Signal Sest Pico Chemiluminescent substrate (Pierce, USA). Synthetic siRNAs were each transfected into FK/R2AN cells and levels of HCV core protein were visualized by Western blot analysis 3 days after transfection (top). The expression levels of -actin were also determined (bottom). Protein lysates were electrophoresed on SDS/15% PAGE gels. Samples from the parental Huh-7 cells (lane 1) and the replicon cells, FK/R2AN (lane 2) are shown. FK/R2AN cells transfected with 80 nM of control siRNA (lane 3) or with 10 nM (lane 4), 20 nM (lane 5), 40 nM (lane 6) or 80 nM (lane 7) of siHCV-7284 were analyzed. The final concentrations were brought up to 80 nM with control siRNA (lanes 4-6).

To confirm the RNAi activity of the selected HCV siRNAs from genomewide siRNA candidates, we examined the levels of reporter protein expression in FK/R2AN cells delivered with synthetic siRNAs by Rluc assay. The 3′ overhang of the chemically synthesized siRNAs corresponding to control siRNA, siHCV-7284 and siHCV-8749 was dTdT, instead of UU, to ensure their stability.

As shown in FIG. 8, the synthetic siRNAs were also inhibited HCV replicon in a dose-dependent manner. By setting the renilla luciferase level by transfection of control siRNA to 100%, inhibition effects of gene expression were 75% by siHCV-7284. It indicates that the silencing activity of a siRNA was more dramatically saturated by using synthetic siRNA than vector-based or PCR product-based siRNA expression DNA.

Previously, it was observed that the pRNAiDu expressing siHCV-7284 siRNA showed significant RNAi effect by reducing the viral protein levels but not the viral RNA transcripts levels. The present inventors supposed that the amount of this siRNA was expressed not enough to inducing saturated gene silencing effects at that detection point. To test this suggestion, the inventors measured the replicon RNA levels by transfection with synthetic siRNAs that guarantees the similar siRNA delivery efficacy into the cytoplasm at the same transfection condition and compared their RNAi effects using real-time RT-PCR (FIG. 9).

Viral RNA levels of the FK/R2AN cells delivered with 80 nM of chemically synthesized negative control siRNA or 10 nM, 20 nM, 40 nM or 80 nM of siHCV-7284 were quantitatively analyzed. The final concentrations were brought up to 80 nM with control siRNA. The HCV RNA values were normalized to -actin RNA values. The numbers of HCV RNA copies per microgram of total RNA were estimated.

Error bars indicate the standard deviations of the averages of three independent experiments.

As shown in FIG. 9, siHCV-7284 also potently reduced the replicon RNA levels in a dose-dependent manner as siHCV-8749 did.

### INDUSTRIAL APPLICABILITY

As explained hereinbefore, the present invention provides a HCV specific siRNA, a DNA vector expressing the siRNA and a pharmaceutical composition for the treatment and prevention of hepatitis C comprising the siRNA or the expression vector as an effective ingredient. The administration of the synthetic siRNA or a DNA molecule encoding the RNA of the invention resulted in the inhibition of HCV gene expression and replication. Therefore, the siRNA and the DNA vector of the present invention can be effectively used for the treatment of hepatitis C virus chronic carrier.

### [Sequence List Text]

Sequences represented by SEQ. ID. NOs: 1-36 are the target nucleotide sequences on the HCV full-length RNA,

Sequences represented by SEQ. ID. NO: 37 and NO: 38 are the U6 forward primer and the H1 forward primer, respectively,

Sequences represented by SEQ. ID. NOs: 39-41 are the HCV 5′UTR specific primer and probe sequences,

Sequences represented by SEQ. ID. NOs: 42-44 are the primer and probe sequences for -actin gene,

Sequences represented by SEQ. ID. NOs: 45-50 are the synthetic sense and anti-sense siRNA sequences corresponding to control siRNA siHCV-7284 and siHCV-8749, respectively.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. An isolated nucleic acid molecule comprising one of the nucleotide sequences of SEQ. ID. NOs: 1-36, or a complement thereof, or a portion thereof.

2. The isolated nucleic acid molecule according to claim 1, which comprises one of the nucleotide sequences of SEQ ID NOs: 4, 10, 16, 18, 19, 23, 29, 33 or 34, or a complement thereof, or a portion thereof.

3. An isolated nucleic acid molecule with hybridization of sense and anti-sense sequences of the nucleic acid molecule of claim 1.

4. The isolated nucleic acid molecule according to claim 3, wherein the nucleic acid molecule is a short interfering RNA (siRNA).

5. The isolated nucleic acid molecule according to claim 3, wherein 3′ ends of the sense strand and anti-sense strand are linked by dTdT molecule.

6. The isolated nucleic acid molecule according to claim 3, wherein the sense strand and anti-sense strand are covalently connected via a linker molecule.

7. The isolated nucleic acid molecule according to claim 5, wherein the linker molecule is a polynucleotide linker.

8. The isolated nucleic acid molecule according to claim 5, wherein the linker molecule is a non-nucleotide linker.

9. The isolated nucleic acid molecule according to claim 1, which binds to the HCV RNA.

10. A DNA vector comprising the nucleotide sequence of claim 1.

11. A DNA vector comprising the nucleotide sequence of claim 2.

12. A pharmaceutical composition for the treatment of HCV infection related disease comprising the nucleic acid molecules of claim 1 as an effective ingredient.

13. The pharmaceutical composition according to claim 12, wherein the composition additionally includes a pharmaceutically acceptable carrier.

14. A treatment method for HCV infection related disease including the step of injecting the nucleic acid molecules of claim 1 into a target subject.

15. An siRNA expression vector, pRNAiDu, represented in FIG. 1 (Accession No: KCTC 10800BP).

16. A pharmaceutical composition for the treatment of HCV infection related disease comprising the nucleic acid molecule of claim 3 as an effective ingredient.

17. A pharmaceutical composition for the treatment of HCV infection related disease comprising the DNA vector of claim 10 as an effective ingredient.

18. A pharmaceutical composition for the treatment of HCV infection related disease comprising the DNA vector of claim 11 as an effective ingredient.

19. A treatment method for HCV infection related disease including the step of injecting the nucleic acid molecule of claim 3 into a target subject.

20. A treatment method for HCV infection related disease including the step of injecting the DNA vector of claim 10 into a target subject.

21. A treatment method for HCV infection related disease including the step of injecting the DNA vector of claim 11 into a target subject.


SEQUENCE LISTING (50 sequences)

SEQ ID NO: 1 - RNA, 19 nt, Artificial Sequence
  HCV siRNA
cuacugucuucacgcagaa

SEQ ID NO: 2 - RNA, 19 nt, Artificial Sequence
  HCV siRNA
agcgucuagccauggcguu

SEQ ID NO: 3 - RNA, 19 nt, Artificial Sequence
  HCV siRNA
cccgcucaaugccuggaga

SEQ ID NO: 4 - RNA, 19 nt, Artificial Sequence
  HCV siRNA
ggcgacaaccuauccccaa

SEQ ID NO: 5 - RNA, 19 nt, Artificial Sequence
  HCV siRNA
ggucaucgauacccucacg

SEQ ID NO: 6 - RNA, 19 nt, Artificial Sequence
  HCV siRNA
ucugcccgguugcuccuuu

SEQ ID NO: 7 - RNA, 19 nt, Artificial Sequence
  HCV siRNA
cguauccggaguguaccau

SEQ ID NO: 8 - RNA, 19 nt, Artificial Sequence
  HCV siRNA
cgcaagcauuguguaugag

SEQ ID NO: 9 - RNA, 19 nt, Artificial Sequence
  HCV siRNA
uauaucccggccacgugac

SEQ ID NO: 10 - RNA, 19 nt, Artificial Sequence
  HCV siRNA
ugacucccucaacacuggg

SEQ ID NO: 11 - RNA, 19 nt, Artificial Sequence
  HCV siRNA
ggcaacugguuuggcugua

SEQ ID NO: 12 - RNA, 19 nt, Artificial Sequence
  HCV siRNA
augggaguauguccuguug

SEQ ID NO: 13 - RNA, 19 nt, Artificial Sequence
  HCV siRNA
ucuugcucgccauacucgg

SEQ ID NO: 14 - RNA, 19 nt, Artificial Sequence
  HCV siRNA
ccaaagugccguacuucgu

SEQ ID NO: 15 - RNA, 19 nt, Artificial Sequence
  HCV siRNA
gguugcugggggucauuau

SEQ ID NO: 16 - RNA, 19 nt, Artificial Sequence
  HCV siRNA
ggcacaugguaucgacccu

SEQ ID NO: 17 - RNA, 19 nt, Artificial Sequence
  HCV siRNA
ugacgggcuuuaccggcga

SEQ ID NO: 18 - RNA, 19 nt, Artificial Sequence
  HCV siRNA
cgagguuacuaccacacac

SEQ ID NO: 19 - RNA, 19 nt, Artificial Sequence
  HCV siRNA
caggcagcguggucauugu

SEQ ID NO: 20 - RNA, 19 nt, Artificial Sequence
  HCV siRNA
agccggccaucauucccga

SEQ ID NO: 21 - RNA, 19 nt, Artificial Sequence
  HCV siRNA
guccuuuaccgggaguucg

SEQ ID NO: 22 - RNA, 19 nt, Artificial Sequence
  HCV siRNA
ucggguugcugcaaacagc

SEQ ID NO: 23 - RNA, 19 nt, Artificial Sequence
  HCV siRNA
gccuucugggcgaagcaua

SEQ ID NO: 24 - RNA, 19 nt, Artificial Sequence
  HCV siRNA
ccuacucccugcuauccuc

SEQ ID NO: 25 - RNA, 19 nt, Artificial Sequence
  HCV siRNA
cauuccccauuaacgcgua

SEQ ID NO: 26 - RNA, 19 nt, Artificial Sequence
  HCV siRNA
gaggacgguuguccuguca

SEQ ID NO: 27 - RNA, 19 nt, Artificial Sequence
  HCV siRNA
ucuaccgugucuucugccu

SEQ ID NO: 28 - RNA, 19 nt, Artificial Sequence
  HCV siRNA
gaaggucaccuuugacaga

SEQ ID NO: 29 - RNA, 19 nt, Artificial Sequence
  HCV siRNA
aggacguccggaaccuauc

SEQ ID NO: 30 - RNA, 19 nt, Artificial Sequence
  HCV siRNA
gccagcucgccuuaucgua

SEQ ID NO: 31 - RNA, 19 nt, Artificial Sequence
  HCV siRNA
gccagacaggccauaaggu

SEQ ID NO: 32 - RNA, 19 nt, Artificial Sequence
  HCV siRNA
accagaauacgacuuggag

SEQ ID NO: 33 - RNA, 19 nt, Artificial Sequence
  HCV siRNA
ggaugauccugaugacuca

SEQ ID NO: 34 - RNA, 19 nt, Artificial Sequence
  HCV siRNA
cggggagcuaaacacucca

SEQ ID NO: 35 - RNA, 19 nt, Artificial Sequence
  HCV siRNA
acacuccaggccaauaggc

SEQ ID NO: 36 - RNA, 19 nt, Artificial Sequence
  HCV siRNA
agguccgugagccgcuuga

SEQ ID NO: 37 - DNA, 22 bp, Artificial Sequence
  U6 forward primer
cggaattccccagtggaaagac

SEQ ID NO: 38 - DNA, 23 bp, Artificial Sequence
  H1 forward primer
cggaattcatatttgcatgtcgc

SEQ ID NO: 39 - DNA, 19 bp, Artificial Sequence
  HCV forward primer
tctgcggaaccggtgagta

SEQ ID NO: 40 - DNA, 23 bp, Artificial Sequence
  HCV backward priemr
attgagcgggttgatccaagaaa

SEQ ID NO: 41 - DNA, 21 bp, Artificial Sequence
  HCV probe
ccggaattgccaggacgaccg

SEQ ID NO: 42 - DNA, 17 bp, Artificial Sequence
  beta-actin forward primer
gcgcggctacagcttca

SEQ ID NO: 43 - DNA, 18 bp, Artificial Sequence
  beta-actin backward primer
tctcgttaatgtcacgat

SEQ ID NO: 44 - DNA, 18 bp, Artificial Sequence
  beta-actin probe
caccacggccgagcggga

SEQ ID NO: 45 - DNA, 21 bp, Artificial Sequence
  control siRNA sense
acuaccguuguuauaggugtt

SEQ ID NO: 46 - DNA, 21 bp, Artificial Sequence
  control siRNA anti-sense
caccuauaacaacgguagutt

SEQ ID NO: 47 - DNA, 21 bp, Artificial Sequence
  siHCV-7284 sense
ggcacaugguaucgacccutt

SEQ ID NO: 48 - DNA, 21 bp, Artificial Sequence
  siHCV-7284 anti-sense
agggucgauaccaugugcctt

SEQ ID NO: 49 - DNA, 21 bp, Artificial Sequence
  siHCV-8749 sense
gccuucugggcgaagcauatt

SEQ ID NO: 50 - DNA, 18 bp, Artificial Sequence
  siHCV-8749 anti-sense
uauucgcccagaaggctt